(19) United States
(12) Patent Application Publication
(10) Pub. No.: US 2016/0374417 A1
(43) Pub. Date: Dec. 29, 2016

(54) HEATED OUTERWEAR

(71) Applicant: E&B Giftware, LLC, Elmsford, NY (US)

(72) Inventors: Paul H. Ross, Pound Ridge, NY (US); Scott Rubenstein, Ridgewood, NJ (US)

(21) Appl. No.: 15/193,730

(22) Filed: Jun. 27, 2016

Related U.S. Application Data
(60) Provisional application No. 62/185,153, filed on Jun. 26, 2015.

(51) Int. Cl. A41D 27/20; A42B 1/24; A41D 23/00; A61F 7/03; A42B 1/00; A43B 7/02; A41D 27/02; A41D 27/28; A41D 19/00; A43B 3/10

(52) U.S. Cl. CPC A41D 27/20; A41D 19/002; A42B 1/241; A41D 23/00; A43B 3/101; A42B 1/008; A43B 7/02; A41D 27/02; A41D 27/28; A61F 7/034; A41D 2400/10; A61F 2007/0088

(57) ABSTRACT

An item of outerwear includes an outer layer that is light weight, water resistant, and wind resistant. An inner layer has a plurality of apertures therethrough connecting an interstitial space between the outer layer and the inner layer with a space interior to the inner layer. A pocket houses a heat pack. A portion of the plurality of apertures permits airflow to and from the heat pack.

### FIELD OF THE APPLICATION

The application relates to outerwear and in particular to outerwear heated by one or more heat packs.

### BACKGROUND

Today's active lifestyles find people outdoors more frequently whether as active participants, or perhaps as spectators of sporting events or children's activities. Regardless, more and better ways of keeping people warm outside for extended periods of time is desirable. Thus, there is a need for outerwear that can withstand the elements and provide warmth for hours at a time.

### SUMMARY

According to one aspect, an item of outerwear includes an outer layer that is light weight, water resistant, and wind resistant. An inner layer has a plurality of apertures therethrough connecting an interstitial space between the outer layer and the inner layer with a space interior to the inner layer. A pocket houses a heat pack. A portion of the plurality of apertures permits airflow to and from the heat pack.

In one embodiment, the pocket is disposed in the interstitial space between the outer layer and the inner layer.

In another embodiment, the pocket is disposed in the space interior to the inner layer.

In yet another embodiment, the outerwear includes a glove.

In yet another embodiment, the outerwear includes a hat.

In yet another embodiment, the outerwear includes a neck warmer.

In yet another embodiment, the outerwear includes a scarf and one side of the scarf comprises a perforated material.

In yet another embodiment, the outerwear includes a slipper.

In yet another embodiment, the outerwear further includes a heat vent.

In yet another embodiment, the heat vent includes a contracting heat vent.

In yet another embodiment, the outerwear further includes a heat-conductive fabric leading from the heat pack to more remote area.

In yet another embodiment, the outerwear further includes a heat-conductive threads leading from the heat pack to a remote area.

In yet another embodiment, the outerwear further includes a heat-conductive fabric or heat-conductive threads leading from the heat pack to a remote area wherein the outerwear includes a glove and the remote area includes a fingertip.

The foregoing and other aspects, features, and advantages of the application will become more apparent from the following description and from the claims.

### BRIEF DESCRIPTION OF THE DRAWINGS

The features of the application can be better understood with reference to the drawings described below, and the claims. The drawings are not necessarily to scale, emphasis instead generally being placed upon illustrating the principles described herein. In the drawings, like numerals are used to indicate like parts throughout the various views.

FIG. 1 shows a drawing of a hat in accordance with some embodiments;

FIG. 2 shows a drawing of a scarf in accordance with some embodiments;

FIG. 3 shows a drawing of a neck warmer in accordance with some embodiments;

FIG. 4 shows a drawing of a glove in accordance with some embodiments;

FIG. 5 shows a drawing of footwear in accordance with some embodiments;

FIG. 6 shows a drawing of footwear in accordance with some embodiments;

FIG. 7 shows a drawing of a hat in accordance with some embodiments;

FIG. 8A shows a drawing of one side of a contemplated heated scarf;

FIG. 8B shows a drawing of the other side of the heated scarf of FIG. 8A.

FIG. 8C shows another view of the contemplated scarf;

FIG. 9A shows a front view of a neck warmer;

FIG. 9B shows an isometric view of the heated neck warmer of FIG. 9A;

FIG. 9C shows an elevated front view of the heated neck warmer of FIG. 9A;

FIG. 10A shows a top view of another heated glove;

FIG. 10B shows a bottom view of the glove of FIG. 10A; and

FIG. 11 shows a drawing of a collection of outerwear in accordance with some embodiments.

### DETAILED DESCRIPTION

Embodiments of the present invention provide for the incorporation of heat packs into various outerwear such as hats, gloves, scarves, neckwarmers, slippers, etc. The outerwear can be provided with a lightweight water and wind resistant outer layer and a perforated inner layer for facilitating airflow into a pocket for housing a heat pack and allow heat dispersion.

Embodiments of the present invention relate to the incorporation of heat packs into various outerwear garments such as but not limited to hats, scarves, gloves, jackets, coats, neck warmers, and the like. By placing a heat pack into a designated pocket or pouch within the item, a user is provided with soothing, targeted, and controlled heat in a consistent, convenient, and comfortable application.

We also described techniques of incorporation of heat packs in co-pending U.S. patent application No. Ser. No. 14/508,628, Air-activated heated travel pillows and travel blankets. In the '628 application, heat packs are incorporated into various travel and comfort items, such as pillows, blankets, and the like, in a designated pocket and/or pouch within the items to provide a user with soothing, targeted, and controlled heat in a consistent, convenient, and comfortable application. A disposable heat pack may comprise a soft, lightweight, air-permeable pouch. One side of the heat pack is air-permeable to optimally direct the targeted heat to a user. The contents of the heat pack may include a mixture of iron powder, sawdust, activated carbon, salt, water, cellulose, and vermiculite. The pocket into which the heat pack is inserted is comprised of a spandex material or the like that is lightweight, permeable, breathable, flexible, and stretchable, providing superior contact while allowing for an optimal amount of heat to permeate from the pocket to the user. The '628 application is incorporated herein by reference in its entirety for all purposes.

Heat packs, also known as body warmers, heat pads, warmers, heating pads, heating packs, hand warmers, foot warmers, toe warmers, pack heat, oxygen-activated heat packs, and/or air-activated chemical warming devices, are known to aid users in keeping parts of the body warm in cold weather environments as well as in heat therapy applications. Depending on the chemical composition of the heat pack, a sustained, controlled heat can be generated for a period of up to 18 hours at a maximum temperature of about 131-170 degrees Fahrenheit. In some embodiments, a heat pack may be disposable. In some embodiments, a heat pack may be safe for air travel.

In one embodiment, the disposable heat pack may comprise a soft, lightweight, air-permeable pouch. In one embodiment, one side of the heat pack is air-permeable to more efficiently provide and optimally direct the targeted heat to a user.

The contents of the heat pack may include, but are not limited to, a mixture of iron powder, sawdust, activated carbon, salt, and water. Other ingredients may include cellulose and vermiculite. A summary of exemplary ingredients and their role in the chemical reaction is provided below:

Iron Powder—Iron is an active ingredient in the heat pack. When the iron combines with oxygen and the other ingredients in the pack, it starts to rust at an accelerated rate. The rusting process is what gives off the heat that warms the heat pack.

Sawdust—Sawdust is used to retain and release moisture in the heat pack, which in turns aids in the dispersion of the heat from the oxidation reaction.

Activated Carbon—Activated carbon may be added to heat packs because it helps absorb the oxygen. Activated Carbon also prevents external odors because unpleasant smelling chemicals released during the oxidation process are captured by it (i.e. stick to it).

Water—Water adds to the reaction within the heat pack as it helps disperse the created heat.

Salt—Salt may be used to accelerate the oxidation process, causing the iron to rust more quickly and generate more heat.

Optional Ingredients—Vermiculite may be added and used as an insulator; it helps the heat pack retain its heat. Vermiculite also absorbs excess moisture. Cellulose may be added as a filler. It holds and releases moisture as well.

In one embodiment, a heat pack uses a specific formulation of iron powder, water, salt, activated carbon, and vermiculite filled to a predetermined maximum weight given their size to provide a quick ramp-up temperature in a comfort temperature zone (e.g. about 55-63 degrees Celsius), and to also provide maintenance of this temperature once reached through an elapsed time of eight hours after initial activation. This composition, based on proportion, size, weight, and heat pack materials, provides a steady heat once the temperature is obtained.

When the heat pack is removed from airtight packaging and exposed to the air, an exothermic oxidation reaction activates, generating controlled and sustained heat from the pouch. Once the iron powder within the heat pack completely oxidizes and the heat pack cools, the pouch will no longer generate heat. For this reason, the heat pack is single use and disposable.

In some embodiments, the outerwear is provided with an exterior fabric that is both lightweight and resistant to wind and water. An inner fabric can be any suitable fabric such as flannel, fleece, cotton, etc. In some embodiments, the inner fabric is perforated, having a plurality of apertures in the fabric to allow airflow to the heat pack and/or to allow heat to pass through the inner layer to the wearer's body. Because heat warmers are introduced, the fabric bulk may be minimized compared to other comparable garments. A breathable pocket for holding a heat pack is formed in and between the outer layer and the inner layer, or between two layers of material other than the outer layer, in some cases between two layers of the inner fabric or in others between the inner fabric and another fabric either on the outer side of the inner fabric or on an interior surface of the inner layer.

The outer fabric is a lightweight material that is both water and wind resistant.

Choice of materials include synthetic fabrics made from polymer materials and blends, such as but not limited to polyamides (such as nylon), polyester, acrylic, rayon, acetate, para-aramids (such as Kevlar™), spandex, latex, Kevlar, and others. Desired properties of light weight, water and wind resistance can be achieved through choice of these materials or via blending, as well as in combination with fabric weave or other fabric making characteristics.

The inner fabric can be any natural or synthetic material or combination thereof. Ideally, the inner fabric provides a measure of insulation, preventing body heat from escaping, balanced with a desire to minimize bulk. Suitable fabrics include cotton, wool, polyester, acrylic, rayon, acetate, para-aramids (such as Kevlar), spandex, latex, Kevlar, and others and combinations thereof. In some embodiments, the inner layer is provided with a plurality of apertures to create a perforated inner layer. The apertures serve at least two distinct functions. First, since it is contemplated that an air activated heat source will be used, the apertures allow for air flow, particularly around the heat source to provide activation. Second, the apertures allow heat to escape the inner layer and to be felt as warmth by the wearer. Proper control of airflow to and from the heat source in useful in extending the useful period of the heatpack and for dissipating heat throughout the garment.

By controlling the amount of airflow to the heat pack, it has been discovered that the useful life of the heat pack can be extended to 8 to 12 hours. Airflow can be controlled to desirable level through a combination of choice of materials, and any or all of the size, shape, number, location, and pattern of perforations provided in the inner layer and/or in the pocket layer. In experiments, certain air-activated heat packs exposed to free access to air, burned themselves out quickly. Not only was the usable life of the heat pack not desirable, but the heat was intensified around the heat pack and would require shielding to protect the wearer. Limiting the airflow to the heat pack by encasing it in a pocket or otherwise between fabric layers, and providing apertures or perforations to allow airflow, achieves the dual benefits of extending the heat packs usable life, and heat distribution while limiting hot spots.

In some embodiments, outerwear in the form of hats and caps, particularly those of the ‘beanie cap’ style are provided. As will be appreciated, caps are particularly well suited to the inclusion of elastic materials in the fabrics to facilitate various head sizes and to allow for a snug fit. In the various hats and caps, an outer shell of lightweight, water and wind resistant material is provided. An inner layer of perforated material is provided. The inner layer may be at least affixed to the outer layer via stitching, adhesive, or other appropriate methods. A lower edge corresponding to the opening for allowing placement on the head may be provided with overlap of one layer onto the other to provide a good closure or to achieve desired aesthetics. That is, the outer layer may be folded under to cover a portion of the inner layer, or vice versa. In some embodiments, each layer may simply be finished at the edge to form a tidy edge without overlap.

Pockets: In some embodiments, the pocket is defined at the interface of two or more materials for housing a heat pack. The two or more materials may be the inner and the outer layer, or the outer layer in combination with a pocket layer for holding the heat pack. The inner layer and any pocket layer are provided with a plurality of apertures to define a perforated fabric to allow appropriate airflow into the pocket. Airflow facilitates the functioning of the heat pack. In some instances, the pocket layer makes up a portion of the inner layer; that is, in those instances, the pocket is formed within the inner layer, or between the outer layer and the inner layer. The perforations also allow the heat generated to dissipate throughout the hat or cap. In some embodiments, channels can be formed between the pocket and other areas of the hat or cap to facilitate heat flow throughout the garment.

Pockets can be defined by existing seams of the structure of the outerwear, or further defined by additional seams. Existing and additional seams include stitching which joins an outer layer to an inner layer. Seams defining pockets can be made by any suitable sewing or stitching techniques. It is contemplated that pockets could also be defined, or further defined about existing seams by any suitable seam technique or layer joining technique, including, for example, glue or adhesive based seams.

Some embodiments include a scarf which is essentially an elongated garment having one side of a lightweight, water resistant and wind resistant fabric and the other a perforated layer. As described above, a pocket is provided either between the layers or defined on the perforated layer. The pocket is adapted for holding a heat pack. The perforated layer and pocket layer are provided with a plurality of apertures which allow for air flow to the heat pack and facilitate heat dispersion through the scarf. Although the heat pack can be placed in any location, the center, near the location corresponding to the back of the neck, or either or both ends of the scarf, or all three locations, are suitable locations.

Some embodiments provide gloves or mittens having a pocket for housing a heat pack. As with other embodiments, an outer lightweight, water and wind resistant fabric is used on the exterior with a perforated insulative layer on the inner surfaces. A pocket is provided, with access via the exterior outer fabric. Gloves are particularly well-suited for the inclusion of means for distributing heat from the heat pack, typically located on the back of the hand or wrist, to the fingers or finger tips. It is possible to include heat-conductive fabric or heat-conductive threads leading from the heat pack to more remote areas such as the finger tips.

Heat Zones:

Heated outerwear includes one or more designated ‘heat zones’ which comprise an area that comes into contact with one or more portions of a user's body. This ‘heat zone’ may feature a sewn-in, or otherwise attached (e.g., glued on), pocket or pouch (e.g., a pocket), which may be fastened at one or more open ends by Velcro, zipper, spandex, elastic, snaps, buttons, or the like. The user activates the heat pack by exposing it to air, and then places it within the pocket on the item. The activated heat pack warms the ‘heat zone’ to provide soothing heat for a sustained period of time in a targeted area during the users trip, when relaxing at home, or whenever used by the user.

The geometry of the pocket is such that the heat pack fits within while having sufficient breathing room and tautness to allow for maximum permeability. Thus, the material of the pocket and the geometry of the pocket provide a ‘heat zone’ with a sustained, targeted, and controlled heat transfer to a specific area of the users body.

In another embodiment, vertical and/or horizontal ribbing may be provided within the pocket to enhance air flow and in particular enhance two-way air flow. The ribbing may be provided in one or more portions of the pocket and on one or both internal sides of the pocket.

In one embodiment, the built-in heat zone comprises a pocket with a heat pack disposed therein. In one embodiment, the built-in heat zone comprises one or more pockets with one or more heat packs disposed within each of the one or more pockets. In another embodiment, the built-in heat zone comprises a plurality of pockets with one or more heat packs disposed within each pocket.

In one embodiment, the heat packs are sized and shaped to fit within a 3″×5″ heat zone (e.g., pocket) without bunching or folding; in this particular embodiment, the size of the heat pack is 2.75″ H×4.50″ W. In another embodiment, the heat pack is roughly 2.75″ H×3.75″ W, with the pocket accordingly sized to receive and contain the heat pack. In other embodiments, if other sized heat packs are utilized, the pocket is sized with a specific alignment and geometry to the heat pack to provide for sufficient breathing room and tautness for maximum permeability.

Examples of Heated Outerwear:

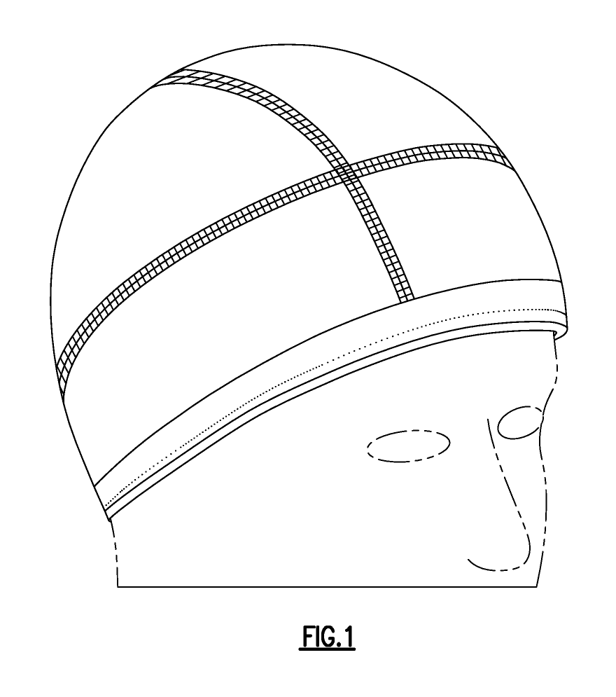

FIG. 1 shows a drawing of an exemplary heated hat. The exemplary hat is made from a TRI-LON™ soft-shell fleece. There is a wind resistant construction for enhanced warmth in breezy conditions. There is also water resistance moisture protection. The ultra-light shell structure is comfortable to wear and easy to store. Concealed pockets, such as according to the pocket techniques described in '628 application, allow heat packs to provide temperatures up to about 150 degrees. Heat ranges from about 131-170 degrees can be used. A set of two heat packs can provide targeted heat for up to about 8 hours (e.g. a range of about 6 to 8 hours).

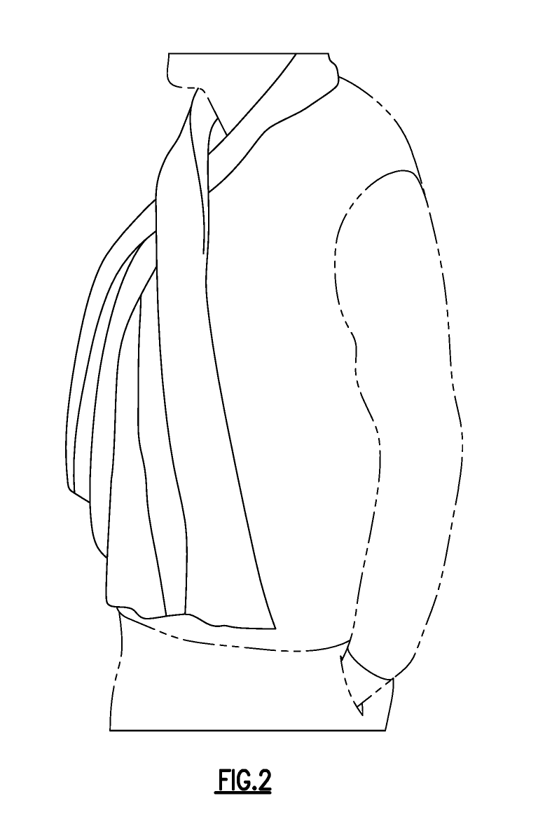

FIG. 2 shows a drawing of an exemplary heated scarf. The exemplary scarf is made from a TRI-LON™ soft-shell fleece. There is a wind resistant construction for enhanced warmth in breezy conditions. There is also water resistance moisture protection. The ultra-light shell structure is comfortable to wear and easy to store. Concealed pockets, such as according to the pocket techniques described in '628 application, allow heat packs to provide temperatures up to about 150 degrees. Heat ranges from about 131-170 degrees can be used. A set of two heat packs can provide targeted heat for up to about 8 hours (e.g. a range of about 6 to 8 hours).

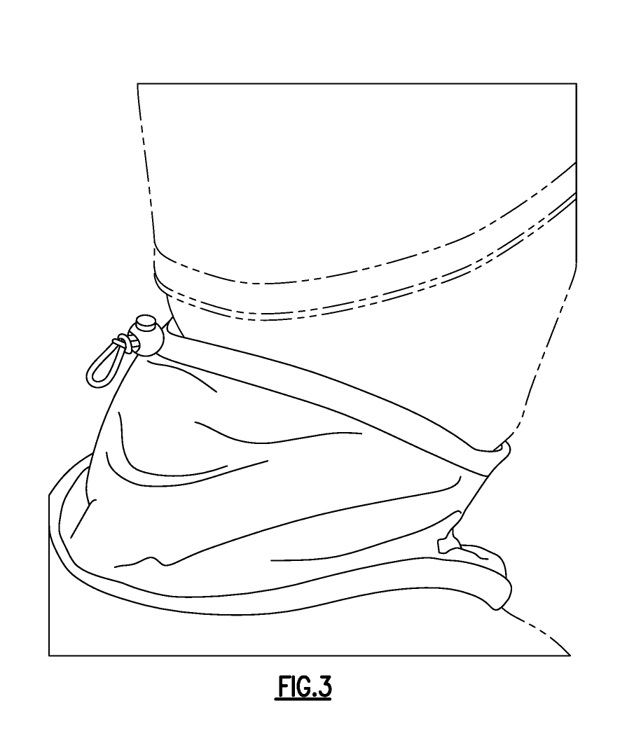

FIG. 3 shows a drawing of an exemplary heated neck warmer. The exemplary neck warmer is made from a TRI-LON™ soft-shell fleece. There is a wind resistant construction for enhanced warmth in breezy conditions. There is also water resistance moisture protection. The ultra-light shell structure is comfortable to wear and easy to store. Concealed pockets, such as according to the pocket techniques described in '628 application, allow heat packs to provide temperatures up to about 150 degrees. Heat ranges from about 131-170 degrees can be used. A set of two heat packs can provide targeted heat for up to about 8 hours (e.g. a range of about 6 to 8 hours).

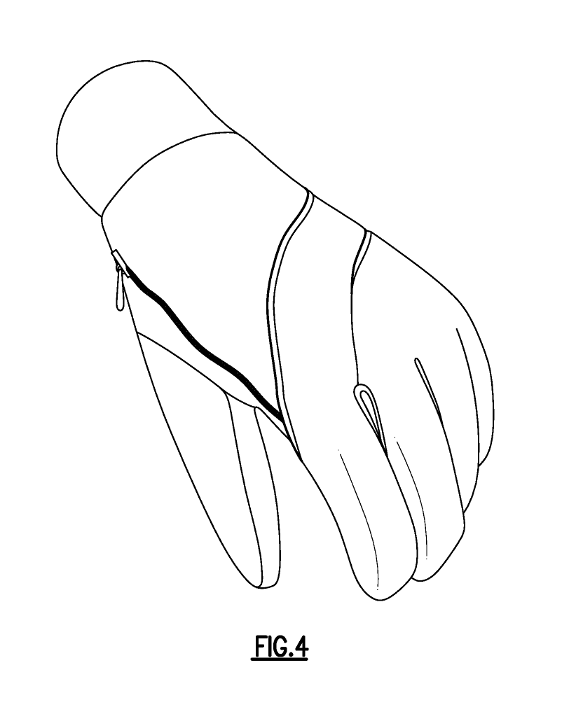

FIG. 4 shows a drawing of an exemplary heated glove. The exemplary heated glove is made from a TRI-LON™ soft-shell fleece. There is a wind resistant construction for enhanced warmth in breezy conditions. There is also water resistance moisture protection. The ultra-light shell structure is comfortable to wear and easy to store. Concealed pockets, such as according to the pocket techniques described in '628 application, allow heat packs to provide temperatures up to about 150 degrees. A zippered heat zone pocket includes a contracting heat vent. Heat ranges from about 131-170 degrees can be used. A set of two heat packs can provide targeted heat for up to about 8 hours (e.g. a range of about 6 to 8 hours).

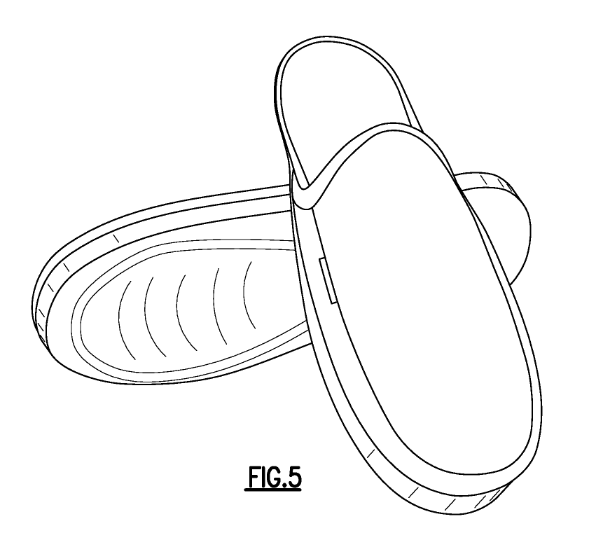

FIG. 5 shows a drawing of an exemplary pair of heated slippers. The exemplary heated slippers are made from a TRI-LON™ DX soft-shell fleece. There is a wind resistant construction for enhanced warmth in breezy conditions. There is also water resistance moisture protection. The ultra-light shell structure is comfortable to wear and easy to store. Concealed pockets, such as according to the pocket techniques described in '628 application, allow heat packs to provide temperatures up to about 150 degrees. Heat ranges from about 131-170 degrees can be used. A set of two heat packs can provide targeted heat for up to about 8 hours (e.g. a range of about 6 to 8 hours).

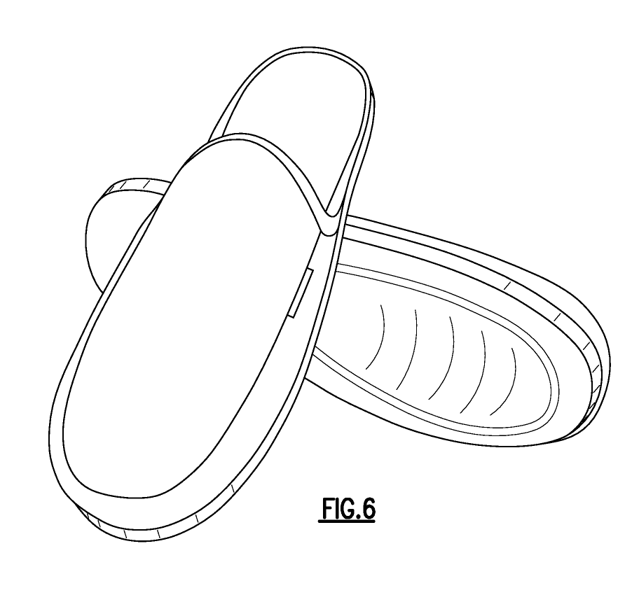

FIG. 6 shows a drawing of another exemplary pair of heated slippers. The exemplary heated slippers are made from a TRI-LON™ DX soft-shell fleece. There is a wind resistant construction for enhanced warmth in breezy conditions. There is also water resistance moisture protection. The ultra-light shell structure is comfortable to wear and easy to store. Concealed pockets, such as according to the pocket techniques described in '628 application, allow heat packs to provide temperatures up to about 150 degrees. Heat ranges from about 120 to 150 degrees can be used. A set of two heat packs can provide targeted heat for up to about 8 hours (e.g. a range of about 6 to 8 hours).

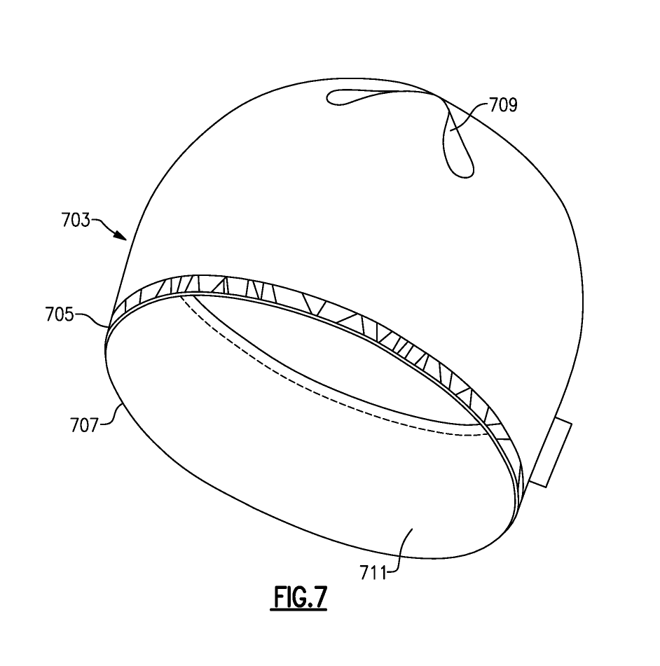

FIG. 7 shows a drawing of another exemplary heated hat, a contemplated ladies' heated hat. The exemplary ladies' hat has an exterior layer of black Polartec™ stretch fleece 703. An exemplary lower trim 705, such as a pink patterned trim provides the structure seem of the bottom hat edge defining the opening for the head. Spandex heat pack pockets 707 can be disposed at any suitable locations with openings (e.g. open pockets, pockets with flaps, zippered pockets, etc.) accessible from either the outside or the inside surface of the hat. For example, one or two pockets can be disposed front and back, or on the right side and the left side of the hat near the opening, approximately at the level of the forehead. The top can be closed by taper seams and/or darts 709 to reduce bulk. The inside of the hat can be finished with a low-pile fleece lining 711.

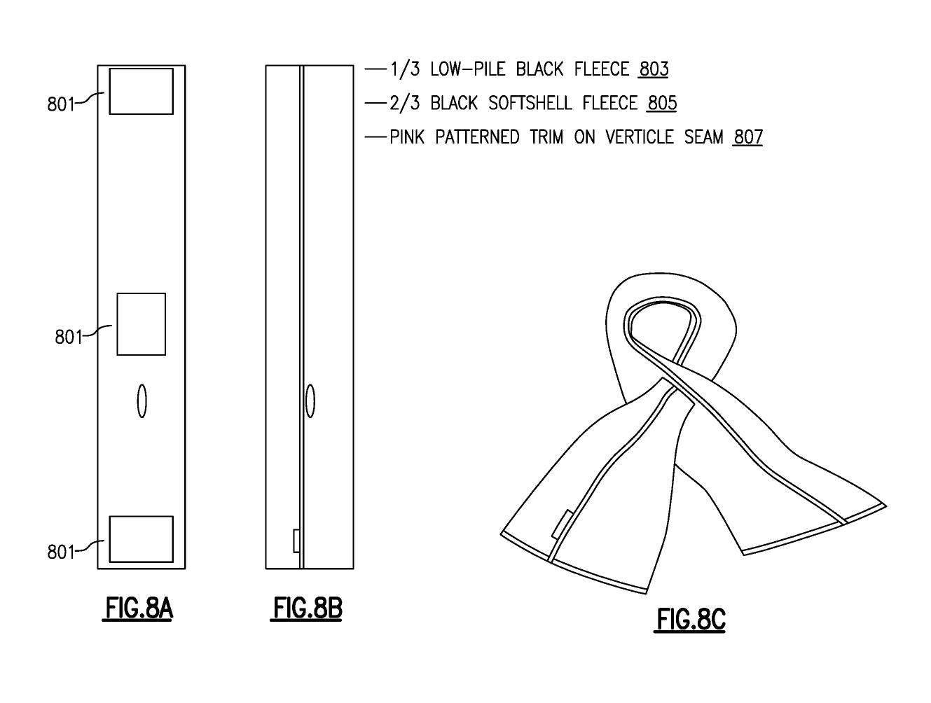

FIG. 8A to FIG. 8C show drawings of another exemplary heated scarf, a contemplated ladies' heated scarf. FIG. 8A shows a drawing of one side of the heated scarf showing spandex heat pack pockets 801. FIG. 8B shows a drawing of the other side of the heated scarf showing a structure of about ⅓ low-pile black fleece 803, about ⅔ black softshell fleece 805, and a pink patterned trim on a vertical seam 807. FIG. 8C shows another view of the contemplated scarf lined with a pink lo-pile fleece fabric with an edge visible slightly over a front seam. Any suitable colors can be used.

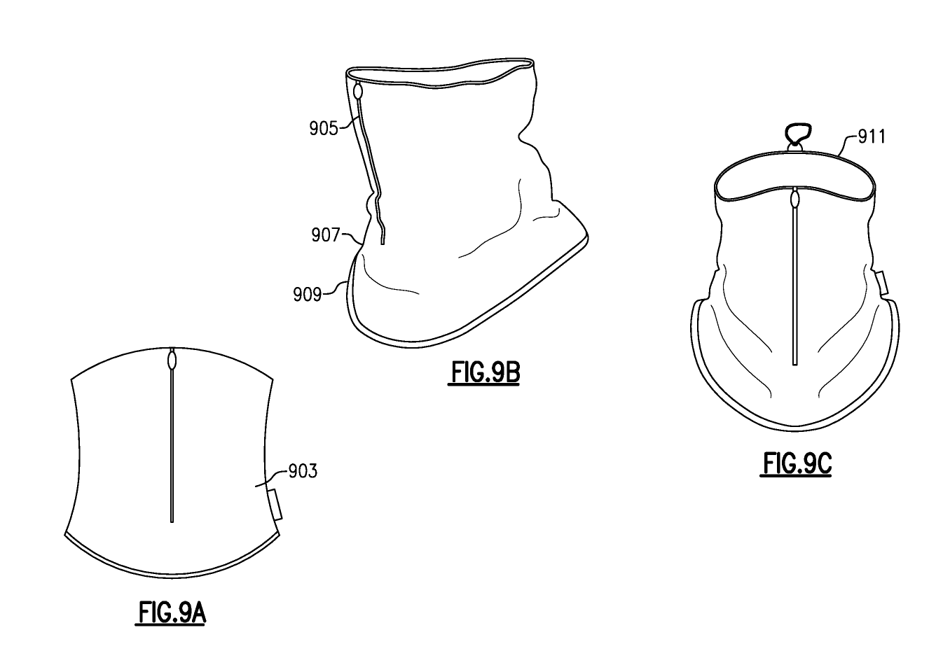

FIG. 9A to FIG. 9C show drawings of a contemplated ladies heated neck warmer. FIG. 9A shows a front view with contour side seams 903 to make for a slimmer fitting over other conventional neck warmers. FIG. 9B shows an isometric view of the heated neck warmer of FIG. 9A. An exemplary ¾ length zipper 905 closed the neck warmer about the neck. The heated neck warmer includes a black soft-shell fleece outer lay 907 and a pink patterned trim 909. FIG. 9C shows an elevated front view of the heated neck warmer of FIG. 9A. The heated neck warmer includes a pink low-pile fleece lining 911. Any suitable colors can be used.

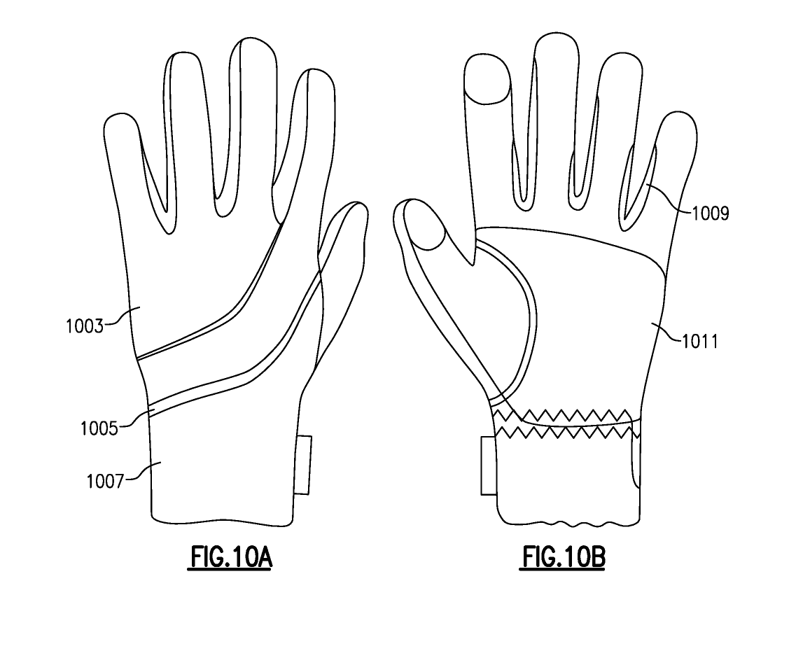

FIG. 10A to FIG. 10B shows drawings of a contemplated ladies heated glove lined with a pink low-pile fabric (heat pack pockets as described by '628 application and hereinabove). FIG. 10A shows a top view of the heated glove. A black softshell fleece outer layer 1003 includes a pink patterned trip 1005. There can be reduced bulk 1007 in the wrist area. FIG. 10B shows a bottom view of the glove of FIG. 10A. There can be a pink softshell fleece 1009 in between the fingers. There can also be a non-slip fabric 1011 on the palm. Any suitable colors can be used.

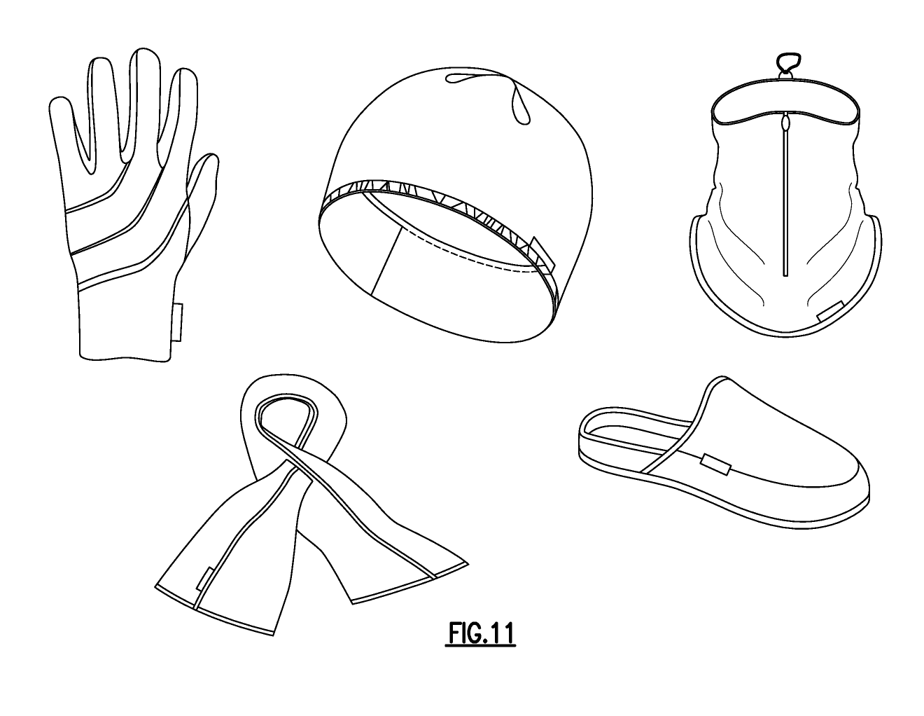

FIG. 11 shows a matching set of the heated glove of FIG. 10A, the heated hat of FIG. 7, the heated neck warmer of FIG. 9A, the heated scarf of FIG. 8A, and the heated slipper of FIG. 5.

Any of the exemplary heated garments can include one or more contracting heat vents. Also, any of the exemplary heated garments can include heat-conductive fabric or heat-conductive threads leading from the heat pack to more remote areas such as, for example, the fingertips of a heated glove.

Similar concepts can be introduced into other outerwear, including but not limited to, shoes and slippers, jackets, vests, coats, sweaters, removable hoods, neck warmers, muffs, etc. Indeed, the concept can be applied to sporting equipment such as helmets, cleats, jerseys, etc. One benefit is that with active heating from the heat pack, the inner layer can be less efficient, since it need not rely on trapping the body's heat but rather on the active heating from the heat pack. Thus, the inner, insulative layer can be less bulky - making for a more aesthetically pleasing look and fit and allowing for more freedom of movement. Alternatively, introduction of the heat packs to a more traditional bulky item, with the perforations, would allow a more traditional garment to be effective at lower temperatures and/or for longer periods of time, since the heat pack provides an additional heat source over the body's own. The introduction of the heat pack also has the advantage of an immediate heating effect. Once the heat pack is activated, active heating is applied, and felt by the user. Traditional garments require time for the body's warmth to be captured and retained within the garment before they feel warm.

Any of the techniques and structures described for a particular embodiment or example can be mixed and matched and/or substituted between embodiments as suitable.

Although the present invention has been described with reference to exemplary embodiments, it is not limited thereto. Those skilled in the art will appreciate that numerous changes and modifications may be made to the preferred embodiments of the invention and that such changes and modifications may be made without departing from the true spirit of the invention. It is therefore intended that the appended claims be construed to cover all such equivalent variations as fall within the true spirit and scope of the invention. For example, the application of various embodiments is not limited to outerwear items and can also be applied to any number of items that provide comfort/relaxation/warmth from cold temperatures in the home, during travel, and the like.

It will be appreciated that variants of the above-disclosed and other features and functions, or alternatives thereof, may be combined into many other different systems or applications. Various presently unforeseen or unanticipated alternatives, modifications, variations, or improvements therein may be subsequently made by those skilled in the art which are also intended to be encompassed by the following claims.

What is claimed is:

1. Outerwear comprising:
an outer layer that is light weight, water resistant, and wind resistant;
an inner layer having plurality of apertures therethrough connecting an interstitial space between the outer layer and the inner layer with a space interior to the inner layer;
a pocket to house a heat pack; and
wherein a portion of the plurality of apertures permits airflow to and from the heat pack.

2. The outerwear of claim 1, wherein said pocket is disposed in said interstitial space between said outer layer and said inner layer.

3. The outerwear of claim 1, wherein said pocket is disposed in said space interior to said inner layer.

4. The outerwear of claim 1, wherein said outerwear comprises a glove.

5. The outerwear of claim 1, wherein said outerwear comprises a hat.

6. The outerwear of claim 1, wherein said outerwear comprises a neck warmer.

7. The outerwear of claim 1, wherein said outerwear comprises a scarf and one side of said scarf comprises a perforated material.

8. The outerwear of claim 1, wherein said outerwear comprises a slipper.

9. The outerwear of claim 1, further comprising a heat vent.

10. The outerwear of claim 9, wherein said heat vent comprises a contracting heat vent.

11. The outerwear of claim 1, further comprising a heat-conductive fabric leading from the heat pack to more remote area.

12. The outerwear of claim 1, further comprising a heat-conductive threads leading from the heat pack to a remote area.

13. The outerwear of claim 1, further comprising a heat-conductive fabric or heat-conductive threads leading from the heat pack to a remote area wherein said outerwear comprises a glove and said remote area comprises a fingertip.
